(19) United States
(12) Patent Application Publication
(10) Pub. No.: US 2018/0021160 A1
(43) Pub. Date: Jan. 25, 2018

(54) KNEE BRACE

(71) Applicant: Fox Head, Inc., Irvine, CA (US)

(72) Inventor: Fong S. Yang, Orange, CA (US)

(73) Assignee: Fox Head, Inc., Irvine, CA (US)

(21) Appl. No.: 15/219,077

(22) Filed: Jul. 25, 2016

(51) Int. Cl. A61F 5/01

(52) U.S. Cl. CPC A61F 5/0123; A61F 2005/0144; A61F 2005/0137; A61F 2005/0179

(57) ABSTRACT

A knee brace includes an upper frame portion, a lower frame portion, a hinge pivotally coupling the upper frame portion and the lower frame portion, and a knee cup. The knee cup includes an outer cup and an inner cup. The outer cup has a first portion coupled to the upper frame portion and a second portion coupled to the lower frame portion. The inner cup has an outer surface and an inner surface. The inner surface is positioned to engage a patella of a wearer of the knee brace.

### BACKGROUND

The subject matter disclosed herein generally relates to a knee brace. More specifically, the present disclosure relates to a knee brace having at least one of (i) a nested knee cup, (ii) a dual pivoting hinge, and (iii) a four bar linkage hinge.

Knee braces may include an upper leg frame and a lower leg frame pivotally coupled together by one or more hinges. The upper leg frame maybe configured to extend above a knee joint and the lower leg frame may be configured to extend below the knee joint. The knee brace may be worn to support, align, or protect the knee of the wearer.

### SUMMARY

One embodiment relates to a knee brace. The knee brace includes an upper frame portion, a lower frame portion, a hinge pivotally coupling the upper frame portion and the lower frame portion, and a knee cup. The knee cup includes an outer cup and an inner cup. The outer cup has a first portion coupled to the upper frame portion and a second portion coupled to the lower frame portion. The inner cup has an outer surface and an inner surface. The inner surface is positioned to engage a patella of a wearer of the knee brace.

Another embodiment relates to a knee brace. The knee brace includes a first frame portion, a second frame portion, and a dual pivoting hinge pivotally coupling the first frame portion and the second frame portion. The dual pivoting hinge includes a first pivot coupling the first frame portion to the dual pivoting hinge, a second pivot coupling the second frame portion to the dual pivoting hinge, and a resilient member extending laterally between the first pivot and the second pivot.

Still another embodiment relates a knee brace. The knee brace includes a first frame portion, a second frame portion, a first coupler, and a second coupler. The first frame portion includes a first extension defining a first hinge interface. The second frame portion includes a second extension defining a second hinge interface. The first coupler extends between the first hinge interface and the second hinge interface. The second coupler extends between the first hinge interface and the second hinge interface. The first hinge interface, the second hinge interface, the first coupler, and the second coupler cooperatively form a four bar linkage that pivotally couples the first frame portion and the second frame portion.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The drawings are provided to illustrate example embodiments described herein and are not intended to limit the scope of the disclosure. Throughout the drawings, reference numbers may be re-used to indicate general correspondence between referenced elements.

FIG. 1 is a front perspective view of a knee brace in a first orientation, according to an exemplary embodiment;

FIG. 2 is a front perspective view of the knee brace of FIG. 1 in a second orientation, according to an exemplary embodiment;

FIG. 3 is a side cross-sectional view of the knee brace of FIG. 1 in the first orientation, according to an exemplary embodiment;

FIG. 4 is a side cross-sectional view of the knee brace of FIG. 2 in the second orientation, according to an exemplary embodiment;

FIG. 5 is a front plan view of the knee brace of FIG. 1 in the first orientation, according to an exemplary embodiment;

FIG. 6 is a side plan view of the knee brace of FIG. 1 in the first orientation, according to another exemplary embodiment;

FIG. 7 is a rear plan view of the knee brace of FIG. 1 in the first orientation, according to an exemplary embodiment;

FIGS. 8-10 are various views of a hinge of the knee brace of FIG. 1 in various orientations, according to an exemplary embodiment;

FIGS. 11-12 are various views of a hinge for a knee brace in various orientations, according to another exemplary embodiment;

FIGS. 13-14 are various views of the hinge of FIGS. 11-12 having a first resilient member, according to an exemplary embodiment;

FIGS. 15-16 are various views of the hinge of FIGS. 11-12 having a second resilient member, according to another exemplary embodiment; and

FIGS. 17-18 are various views of the hinge of FIGS. 11-12 having a third resilient member, according to still another exemplary embodiment.

### DETAILED DESCRIPTION

Various aspects of the disclosure will now be described with regard to certain examples and embodiments, which are intended to illustrate but not to limit the disclosure. Nothing in this disclosure is intended to imply that any particular feature or characteristic of the disclosed embodiments is essential. The scope of protection is defined by the claims that follow this description and not by any particular embodiment described herein. Before turning to the figures, which illustrate example embodiments in detail, it should be understood that the application is not limited to the details or methodology set forth in the description or illustrated in the figures. It should also be understood that the terminology is for the purpose of description only and should not be regarded as limiting.

Embodiments herein generally relate to a knee brace. The knee brace may be configured to protect a knee, thigh, and/or shin of a wearer of the knee brace from impacts with flying debris (e.g., rocks, dirt, mud, etc.), during a collision or crash, and/or during a fall. Such a knee brace may be used in a number of activities, including without limitation: sports and athletics, including extreme sports such as motocross and snowmobiling; cycling activities, including auto racing, motorcycle riding and racing, BMX, mountain biking, etc.; with recreational vehicles including all-terrain vehicles (ATVs), dirt bikes, utility task vehicles (UTVs), snowmobiles, and other off-road vehicles; military applications; and/or construction applications to name just a few. Further details are provided herein.

According to an exemplary embodiment, the knee brace of the present disclosure includes at least one of (i) a nested knee cup, (ii) a dual pivoting hinge, and (iii) a four bar linkage hinge. Traditional dual pivoting hinges of knee braces may include a resilient member arranged in a “figure eight” around two pivot points. During operation of such knee braces, the ligaments of the resilient member cross each other, thereby causing friction therebetween which may cause inefficiencies in rebound of the hinge and premature wear of the resilient member due to the contact. Additionally, the ligaments of figure eight resilient members need to be limited in thickness such that the figure eight resilient members have sufficient space within the hinge to cross over each other, which may adversely impact the strength and tuning capability of the biasing forces. Further, figure eight resilient member may provide identical biasing forces in both flexion and extension. According to an exemplary embodiment, the dual pivoting hinge of the present disclosure includes resilient members that extend laterally between the two pivot points. The lateral arrangement may advantageously (i) prevent inefficiencies in rebound and premature wear relative to traditional figure eight resilient members, (ii) provide more available space within the dual pivoting hinge to increase the size of the resilient member to increase rebound or rotational bias in flexion and/or extension relative to traditional dual pivoting hinges, and/or (iii) facilitate independently tuning the rebound characteristics of the dual pivoting hinge in flexion and extension.

### Knee Brace

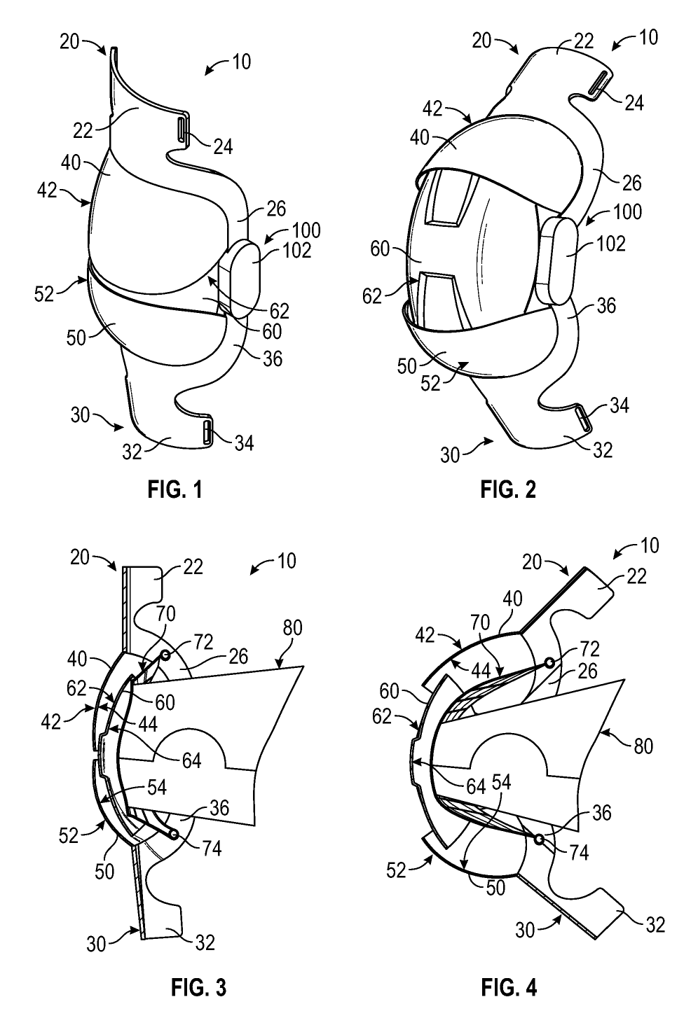

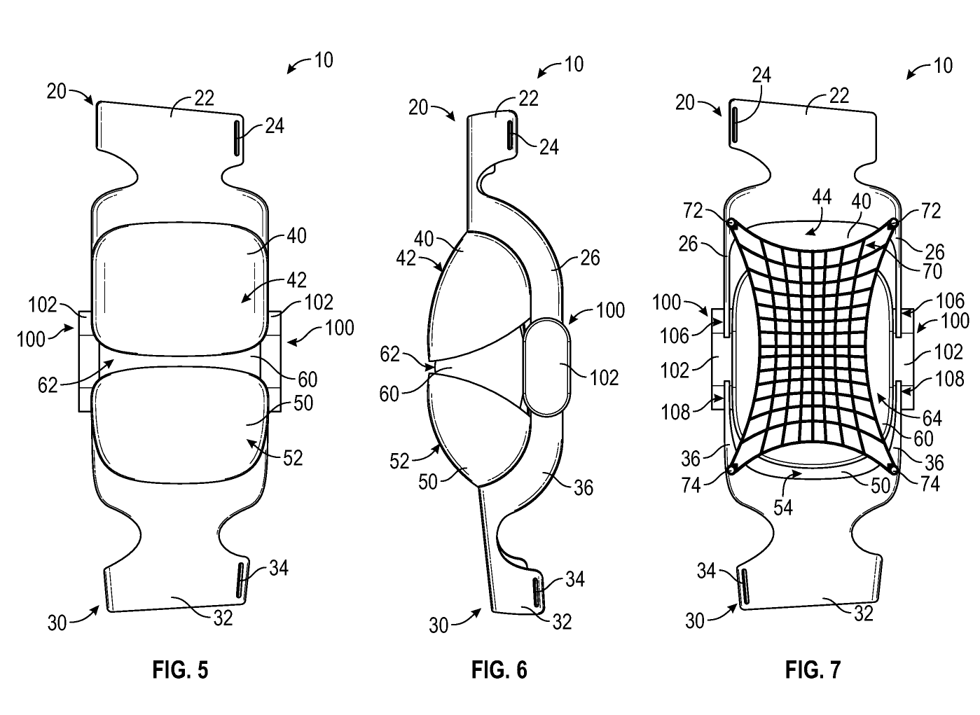

According to the exemplary embodiment shown in FIGS. 1-18, a knee brace, shown as knee brace 10, includes: a frame having a first frame portion, shown as upper frame member 20, and a second frame portion, shown as lower frame member 30; a knee cup (e.g., a nested knee cup, etc.) including (i) a first, outer cup having first portion, shown as upper cup portion 40, and a second portion, shown as lower cup portion 50, and (ii) a second, inner cup, shown as inner cup 60; and at least one of (i) a first hinge system including at least one first hinge, shown as dual pivoting hinges 100, and (ii) a second hinge system including at least one second hinge, shown as four bar linkage hinges 200, pivotally coupling the upper frame member 20 and the lower frame member 30.

As shown in FIGS. 1-7, the upper frame member 20 includes an upper body, shown as upper cuff 22, and a pair of extensions, shown as upper arms 26, extending therefrom. According to an exemplary embodiment, the upper cuff 22 is configured (e.g., sized, shaped, positioned, etc.) to extend above a knee and correspond with an upper leg (e.g., a thigh, a quadricep, etc.) of a wearer of the knee brace 10. As shown in FIGS. 1-2 and 5-7, the upper cuff 22 defines an aperture, shown as upper aperture 24. In some embodiments, the upper frame member 20 includes an upper strap or band configured to facilitate selectively coupling the upper frame member 20 to the upper leg of the wearer of the knee brace 10. According to an exemplary embodiment, the upper aperture 24 is configured to facilitate selectively adjusting the size of the upper strap or band to allow the upper frame member 20 to be coupled to various sized upper legs of various wearers of the knee brace 10.

As shown in FIGS. 1-7, the lower frame member 30 includes a body, shown as lower cuff 32, and a pair of extensions, shown as lower arms 36, extending therefrom. According to an exemplary embodiment, the lower cuff 32 is configured (e.g., sized, shaped, positioned, etc.) to extend below the knee and correspond with a lower leg (e.g., a shin, a calf, etc.) of the wearer of the knee brace 10. As shown in FIGS. 1-2 and 5-7, the lower cuff 32 defines an aperture, shown as lower aperture 34. In some embodiments, the lower frame member 30 includes a lower strap or band configured to facilitate selectively coupling the lower frame member 30 to the lower leg of the wearer of the knee brace 10. According to an exemplary embodiment, the lower aperture 34 is configured to facilitate selectively adjusting the size of the lower strap or band to allow the lower frame member 30 to be coupled to various sized lower legs of various wearers of the knee brace 10.

As shown in FIGS. 1-7, the upper cup portion 40 of the outer cup is coupled to the upper frame member 20. The upper cup portion 40 may thereby pivot with the upper frame member 20 about the dual pivoting hinges 100 (and/or the four bar linkage hinges 200). As shown in FIGS. 1-7, the upper cup portion 40 has a first side, shown as exterior surface 42, and an opposing second side, shown as interior surface 44. As shown in FIGS. 1-7, the lower cup portion 50 of the outer cup is coupled to the lower frame member 30. The lower cup portion 50 may thereby pivot with the lower frame member 30 about the dual pivoting hinges 100 (and/or the four bar linkage hinges 200). As shown in FIGS. 1-7, the lower cup portion 50 has a first side, shown as exterior surface 52, and an opposing second side, shown as interior surface 54.

As shown in FIGS. 1-2 and 5-7, the inner cup 60 is coupled to the dual pivoting hinges 100 (and/or the four bar linkage hinges 200). As shown in FIGS. 1-7, the inner cup 60 includes a first side, shown as outer surface 62, and an opposing second side, shown as inner surface 64. As shown in FIGS. 3-4, the interior surface 44 of the upper cup portion 40 of the outer cup rotates along the outer surface 62 of the inner cup 60 as the upper frame member 20 pivots about the dual pivoting hinges 100 (and/or the four bar linkage hinges 200) and the inner surface 64 of the lower cup portion 50 of the outer cup rotates along the outer surface 62 of the inner cup 60 as the lower frame member 30 pivots about the dual pivoting hinges 100 (and/or the four bar linkage hinges 200). As shown in FIGS. 1,3, and 5-6, the upper cup portion 40 and the lower cup portion 50 of the outer cup enclose around the inner cup 60 when the knee brace 10 is arranged in an extended orientation (and/or a nominal/neutral orientation). As shown in FIGS. 2 and 4, the upper cup portion 40 and the lower cup portion 50 of the outer cup separate to expose the inner cup 60 when the knee brace 10 is arranged in a flexed orientation. The inner cup 60 may thereby remain stationary relative to the outer cup as (i) the upper frame member 20 and the upper cup portion 40 and/or (ii) the lower frame member 30 and the lower cup portion 50 pivot about the dual pivoting hinges 100 (and/or the four bar linkage hinges 200).

According to an exemplary embodiment, the inner surface 64 of the inner cup 60 is configured (e.g., positioned, sized, shaped, etc.) to engage a patella (i.e., a knee cap) of the wearer of the knee brace 10. In some embodiments, the inner surface 64 is always in contact with the patella of the wearer of the knee brace 10. In some embodiments, the inner surface 64 is spaced a distance from the patella of the wearer of the knee brace 10 and may engage the patella during impacts. In some embodiments, the inner surface 64 of the inner cup 60 includes a padding liner or material to assist in impact absorption (e.g., mitigation, reduction, attenuation, etc.). The nested knee cup arrangement of the upper cup portion 40, the lower cup portion 50, and the inner cup 60 may thereby provide impact protection (e.g., from flying debris, during a collision or crash, during a fall, etc.) while the knee brace 10 is arranged in the extended orientation or the nominal/neutral orientation (e.g., during extension of the wearer's knee, during no flexion or extension of the wearer's knee, etc.) and the inner cup 60 may provide impact protection (e.g., from flying debris, during a collision or crash, during a fall, etc.) while the knee brace 10 is arranged in the flexed orientation (e.g., during flexion of the wearer's knee, etc.).

As shown in FIGS. 3-4 and 7, the knee brace 10 includes a patella immobilization elastomer (PIE), shown as PIE liner 70. The PIE liner 70 includes a first end, shown as upper end 72, coupled to the upper frame member 20 and an opposing second end, shown as lower end 74, coupled to the lower frame member 30. The PIE liner 70 may thereby extend across the inner surface 64 of the inner cup 60 such that the PIE liner 70 is positioned to engage the patella of the wearer of the knee brace 10. According to an exemplary embodiment, the PIE liner 70 is positioned to maintain engagement with the patella of the wearer of the knee brace 10 throughout the entire motion of the knee brace 10 (e.g., from full extension to full flexion, providing a more secure fit and feel to the wearer, etc.). According to an exemplary embodiment, the PIE liner 70 is configured to assist in securing the patella of the wearer of the knee brace 10 in place and/or within the inner cup 60.

Traditional knee impact pads may be uncomfortably hot, limit motion, and/or protect only against impacts, while failing to stabilize the patella. The PIE liner 70 may provide a cooling functionality, enable natural movement of the patella, and/or protect against impacts, as well as stabilize the patella. According to an exemplary embodiment, the PIE liner 70 is configured to mimic kinesiology tape (e.g., to aid in affecting a desired biomechanical movement of the patella, etc.). As shown in FIGS. 3-4 and 7, the PIE liner 70 includes netting that is configured to be pulled up against the patella of the wearer of the knee brace 10. The netting inherently provides a breathable/cooling functionality. In one embodiment, the netting has a constant density weave. In other embodiments, the netting has a variable density weave. The variable density weave may be tuned to the geometry of the patella. In another embodiment, the PIE liner 70 additionally or alternatively includes a four-way stretch fabric material. The four-way stretch fabric material may be breathable. In yet another embodiment, the PIE liner 70 additionally or alternatively includes a Jacquard weave combining various different materials. The Jacquard weave may (i) create a desired stretch pattern, (ii) create a desired immobilization pattern, and/or (iii) create a desired ventilation pattern.

As shown in FIGS. 3-4, the knee brace 10 includes a sleeve, shown as patella sleeve 80. The patella sleeve 80 may be configured to receive a leg of the wearer of the knee brace 10 and engage the knee region of the wearer. The patella sleeve 80 may provide additional support and stabilization for the patella of the wearer of the knee brace 10. In other embodiments, the knee brace 10 does not include the patella sleeve 80.

### Dual Pivoting Hinge

According to the exemplary embodiment shown as in FIGS. 1-7, the knee brace 10 includes a pair of dual pivoting hinges 100, a first dual pivoting hinge 100 positioned on a lateral side (e.g., an exterior side, an outer side, etc.) of the knee brace 10 and a second dual pivoting hinge 100 positioned on a medial side (e.g., an inner side, an interior side, etc.) of the knee brace 10. Two dual pivoting hinges 100 may reduce the mechanical loading on each of the dual pivoting hinges 100 of the knee brace 10. In other embodiments, the knee brace 10 includes a single dual pivoting hinge 100 positioned on the lateral side of the knee brace 10. The single dual pivoting hinge 100 may provide a lower profile design on the medial side of the knee brace 10 facilitating the wearer of the knee brace 10 to have closer contact with a vehicle (e.g., a motorcycle, dirt bike, bicycle, etc.) to provide enhanced control, feel, etc. In still other embodiments, the knee brace 10 includes a single dual pivoting hinge 100 positioned on the medial side of the knee brace 10.

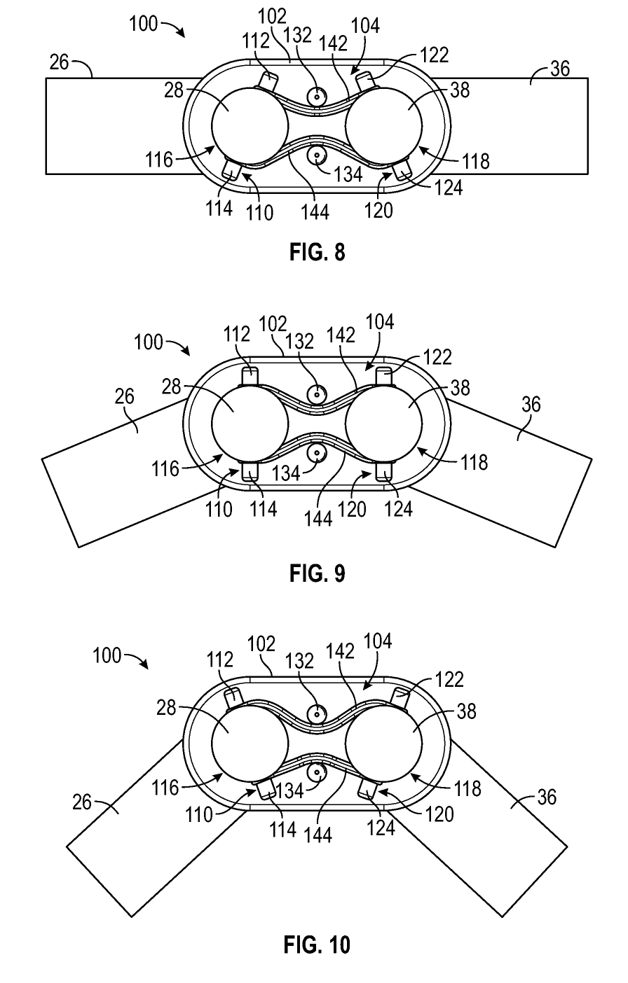

As shown in FIGS. 1-2 and 5-10, each of the dual pivoting hinges 100 includes a body, shown as housing 102. As shown in FIG. 7, each of the housings 102 defines a pair of apertures, shown as upper aperture 106 and lower aperture 108. The upper apertures 106 are configured to receive the upper arms 26 of the upper frame member 20 and the lower apertures 108 are configured to receive the lower arms 36 of the lower frame member 30. As shown in FIGS. 8-10, the housing 102 of the dual pivoting hinges 100 defines an internal cavity, shown as cavity 104. The cavity 104 is configured to receive various components that control the operation and functionality of the dual pivoting hinges 100.

As shown in FIGS. 8-10, each of the upper arms 26 of the upper frame member 20 is pivotally coupled to a respective housing 102 and rotates about a first joint, shown as upper pivot 116. Each of the upper pivots 116 is configured to receive a first coupler, shown as upper coupler 28, that extends through the upper pivot 116 from the cavity 104 to a respective upper arm 26, thereby pivotally coupling the upper frame member 20 to the dual pivoting hinge 100. As shown in FIGS. 8-10, each of the lower arms 36 of the lower frame member 30 is pivotally coupled to a respective housing 102 and rotates about a second joint, shown as lower pivot 118. Each of the lower pivots 118 is configured to receive a second coupler, shown as lower coupler 38, that extends from the cavity 104 through the upper pivot 116 to a respective lower arm 36, thereby pivotally coupling the lower frame member 30 to the dual pivoting hinge 100.

As shown in FIGS. 8-10, the dual pivoting hinges 100 include a first retainer, shown as upper pin 110, extending through the upper coupler 28. The upper pin 110 has a first end, shown as flexion end 112, and an opposing second end, shown as extension end 114. As shown in FIGS. 8-10, the dual pivoting hinges 100 include a second retainer, shown as lower pin 120, extending through the lower coupler 38. The lower pin 120 has a first end, shown as flexion end 122, and an opposing second end, shown as extension end 124. As shown in FIGS. 8-10, the dual pivoting hinges 100 include a third retainer, shown as flexion tensioner 132, and a fourth retainer, shown as extension tensioner 134.

As shown in FIGS. 8-10, the dual pivoting hinges 100 include a first resilient member, shown as flexion elastomer band 142, and a second resilient member, shown as extension elastomer band 144. The flexion elastomer band 142 extends laterally between the flexion end 112 of the upper pin 110 and the flexion end 122 of the lower pin 120. According to an exemplary embodiment, the flexion tensioner 132 is positioned to engage the flexion elastomer band 142 to maintain tension in the flexion elastomer band 142 such that the flexion elastomer band 142 does not inadvertently disengage from the flexion end 112 of the upper pin 110 and/or the flexion end 122 of the lower pin 120. The extension elastomer band 144 extends laterally between the extension end 114 of the upper pin 110 and the extension end 124 of the lower pin 120. According to an exemplary embodiment, the extension tensioner 134 is positioned to engage the extension elastomer band 144 to maintain tension in the extension elastomer band 144 such that the extension elastomer band 144 does not inadvertently disengage from the extension end 114 of the upper pin 110 and/or the extension end 124 of the lower pin 120.

As shown in FIG. 9, the dual pivoting hinge 100 is arranged in a nominal or neutral orientation. In the nominal or neutral orientation, the flexion elastomer band 142 and the extension elastomer band 144 may be in equilibrium (e.g., the flexion elastomer band 142 and the extension elastomer band 144 are not biasing the upper frame member 20 and/or the lower frame member 30 towards another orientation, etc.).

As shown in FIG. 8, the dual pivoting hinge 100 is arranged in an extended orientation. In the extended orientation, the upper frame member 20 and/or the lower frame member 30 pivot about the dual pivoting hinge 100 such that the flexion end 112 of the upper pin 110 and the flexion end 122 of the lower pin 120 rotate towards each other, while the extension end 114 of the upper pin 110 and the extension end 124 of the lower pin 120 rotate away from each other (e.g., in response to the wearer of the knee brace 10 extending/straightening his/her leg, etc.). Such rotation pulls on the extension elastomer band 144, while relaxing the flexion elastomer band 142. The pulling of the extension elastomer band 144 causes the extension elastomer band 144 to provide a biasing force against the rotation of the upper frame member 20 and/or the lower frame member 30 to rotationally bias the dual pivoting hinge 100 and the knee brace 10 back towards the nominal or neutral orientation (e.g., see FIG. 9, etc.). In some embodiments, the housing 102 of the dual pivoting hinges 100 includes a first stop positioned to prevent hyperextension of the knee of the wearer of the knee brace 10.

As shown in FIG. 10, the dual pivoting hinge 100 is arranged in a flexed orientation. In the flexed orientation, the upper frame member 20 and/or the lower frame member 30 pivot about the dual pivoting hinge 100 such that the flexion end 112 of the upper pin 110 and the flexion end 122 of the lower pin 120 rotate away from each other, while the extension end 114 of the upper pin 110 and the extension end 124 of the lower pin 120 rotate towards each other (e.g., in response to the wearer of the knee brace 10 flexing/bending his/her leg, etc.). Such rotation pulls on the flexion elastomer band 142, while relaxing the extension elastomer band 144. The pulling of the flexion elastomer band 142 causes the flexion elastomer band 142 to provide a biasing force against the rotation of the upper frame member 20 and/or the lower frame member 30 to rotationally bias the dual pivoting hinge 100 and the knee brace 10 back towards the nominal or neutral orientation (e.g., see FIG. 9, etc.). In some embodiments, the housing 102 of the dual pivoting hinges 100 includes a second stop positioned to prevent hyper-flexion of the knee of the wearer of the knee brace 10.

In one embodiment, the flexion elastomer band 142 and the extension elastomer band 144 have similar characteristics (e.g., spring constant, biasing force, length, etc.) such that the dual pivoting hinges 100 may bias the knee brace 10 similarly during extension and flexion. In other embodiments, the flexion elastomer band 142 and the extension elastomer band 144 have different characteristics (e.g., spring constant, biasing force, length, etc.) such that the dual pivoting hinges 100 may bias the knee brace 10 differently during extension and flexion. In some embodiments, the flexion elastomer band 142 and the extension elastomer band 144 are asymmetrically loaded to adjust the neutral position (e.g., the neutral angle, etc.) of the knee brace 10 in the neutral orientation. By way of example, the flexion elastomer band 142 and/or the extension elastomer band 144 may be configured such that the knee brace 10 is naturally biased toward a neutral orientation that is slightly flexed.

In some embodiments, the dual pivoting hinges 100 include a plurality of flexion elastomer bands 142 and/or a plurality of extension elastomer bands 144 to selectively adjust (e.g., increase, decrease, etc.) the biasing force during flexion and/or extension of the knee brace 10. In some embodiments, the dual pivoting hinges 100 do not include the flexion elastomer bands 142 such that the dual pivoting hinges 100 encourage flexion (i.e., the dual pivoting hinges 100 bias against extension, not flexion). In other embodiments, the dual pivoting hinges 100 do not include the extension elastomer bands 144 such that the dual pivoting hinges 100 encourage extension (i.e., the dual pivoting hinges 100 bias against flexion, not extension).

In some embodiments, one of the two dual pivoting hinges 100 (e.g., in a two hinge embodiment, etc.) of the knee brace 10 does not include the flexion elastomer band 142 and the extension elastomer band 144. For example, a dual pivoting hinge 100 positioned on the medial side of the knee brace 10 may not include the flexion elastomer band 142 and the extension elastomer band 144 such that the dual pivoting hinge 100 may have a lower profile (e.g., relative to the dual pivoting hinge 100 having the flexion elastomer band 142 and/or the extension elastomer band 144, etc.). The lower profile of the dual pivoting hinge 100 may provide the wearer of the knee brace 10 with closer contact with a vehicle (e.g., motorcycle, dirt bike, ATV, etc.) he/she is driving.

According to an exemplary embodiment, the isolated, lateral arrangement of the flexion elastomer band 142 and the extension elastomer band 144 advantageously prevents inefficiencies in rebound and premature wear relative to traditional figure eight resilient members. Further, the flexion elastomer band 142 and the extension elastomer band 144 have more available space within the cavity 104 relative to figure eight resilient members such that the thickness of the flexion elastomer band 142 and/or the extension elastomer band 144 may be increased. Such an increase may allow the dual pivoting hinge 100 to provide increased rebound or rotational bias in flexion and/or extension relative to traditional dual pivoting hinges. Additionally, isolating the flexion elastomer band 142 and the extension elastomer band 144 relative to each other facilitates independently tuning the rebound characteristics of the dual pivoting hinge 100 in flexion and extension (e.g., the dual pivoting hinge 100 may be tuned to bias more against extension than flexion, etc.).

### Four Bar Linkage Hinge

According to an exemplary embodiment, the knee brace 10 additionally or alternatively includes at least one four bar linkage hinge 200. In one embodiment, the knee brace 10 includes a first four bar linkage hinge 200 positioned on a lateral side (e.g., an exterior side, an outer side, etc.) of the knee brace 10 and a second four bar linkage hinge 200 positioned on a medial side (e.g., an inner side, an interior side, etc.) of the knee brace 10. Two four bar linkage hinges 200 may reduce the mechanical loading on each of the four bar linkage hinges 200 of the knee brace 10. In other embodiments, the knee brace 10 includes a single four bar linkage hinge 200 positioned on the lateral side of the knee brace 10. The single four bar linkage hinge 200 may provide a lower profile design on the medial side of the knee brace 10 facilitating the wearer of the knee brace 10 to have closer contact with a vehicle (e.g., a motorcycle, dirt bike, bicycle, etc.) to provide enhanced control, feel, etc. In still other embodiments, the knee brace 10 includes a single four bar linkage hinge 200 positioned on the medial side of the knee brace 10. In another embodiment, the knee brace 10 includes at least one of (i) the four bar linkage hinge 200 and (ii) the dual pivoting hinge 100.

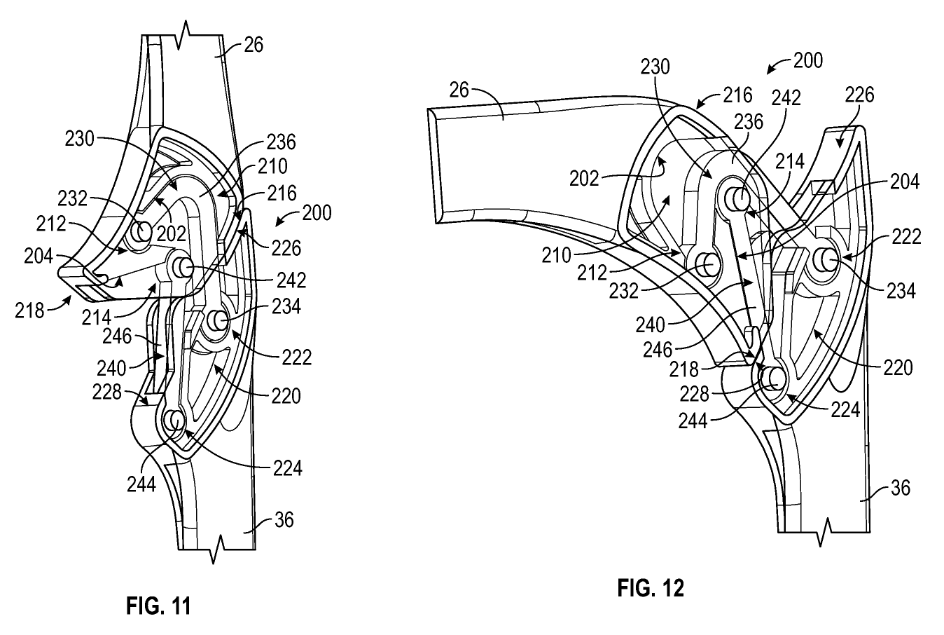

As shown in FIGS. 11-18, each of the upper arms 26 of the upper frame member 20 defines a first hinge interface, shown as upper hinge interface 210, positioned at an end thereof and each of the lower arms 36 of the lower frame members 30 defines a second hinge interface, shown as lower hinge interface 220, positioned at an end thereof. As shown in FIGS. 11-12, the upper hinge interface 210 defines a first joint, shown as first upper pivot 212, and a second joint, shown as second upper pivot 214. The lower hinge interface 220 defines a third joint, shown as first lower pivot 222, and a fourth joint, shown as second lower pivot 224.

As shown in FIGS. 11-12, the four bar linkage hinge 200 includes a first coupler, shown as first connector 230. The first connector 230 includes a body, shown as first link 236, having a first end, shown as upper end 232, and an opposing second end, shown as lower end 234. The upper end 232 of the first link 236 is coupled to the first upper pivot 212 of the upper hinge interface 210 and the lower end 234 of the first link 236 is coupled to the first lower pivot 222 of the lower hinge interface 220 such that the first connector 230 extends between the upper hinge interface 210 and the lower hinge interface 220.

As shown in FIGS. 11-12, the four bar linkage hinge 200 includes a second coupler, shown as second connector 240. The second connector 240 includes a body, shown as second link 246, having a first end, shown as upper end 242, and an opposing second end, shown as lower end 244. The upper end 242 of the second link 246 is coupled to the second upper pivot 214 of the upper hinge interface 210 and the lower end 244 of the second link 246 is coupled to the second lower pivot 224 of the lower hinge interface 220 such that the second connector 240 extends between the upper hinge interface 210 and the lower hinge interface 220.

According to an exemplary embodiment, the upper hinge interface 210, the lower hinge interface 220, the first connector 230, and the second connector 240 cooperatively form a four bar linkage that pivotally couples the upper frame member 20 and the lower frame member 30 together. By way of example, the first linkage may be the first link 236, the second linkage may be formed between the upper end 232 of the first link 236 and the upper end 242 of the second link 246, the third linkage may be the second link 246, and the fourth linkage may be formed between the lower end 244 of the second link 246 and the lower end 234 of the first link 236. The four bar linkage of the four bar linkage hinges 200 is configured to mimic the natural movement of the knee of the wearer of the knee brace 10, according to an exemplary embodiment.

As shown in FIGS. 11-12, the upper hinge interface 210 of the upper arm 26 includes or defines a first limiter, shown as first upper stop 216, positioned at a front end of the upper hinge interface 210 and the lower hinge interface 220 of the lower arm 36 includes or defines a second limiter, shown as first lower stop 226, positioned at a front end of the lower hinge interface 220. As shown in FIG. 11, the first upper stop 216 and the first lower stop 226 are positioned to engage with one another to limit an amount of extension of the knee brace 10. Such limiting of the extension of the knee brace 10 may assist in preventing hyperextension of the knee of the wearer of the knee brace 10.

As shown in FIGS. 11-12, the upper hinge interface 210 of the upper arm 26 includes or defines a third limiter, shown as second upper stop 218, positioned at a rear end of the upper hinge interface 210 and the lower hinge interface 220 of the lower arm 36 includes or defines a fourth limiter, shown as second lower stop 228, positioned at a rear end of the lower hinge interface 220. As shown in FIG. 12, the second upper stop 218 and the second lower stop 228 are positioned to engage with one another to limit an amount of flexion of the knee brace 10. Such limiting of the flexion of the knee brace 10 may assist in preventing hyper-flexion of the knee of the wearer of the knee brace 10.

As shown in FIGS. 11-12, the upper hinge interface 210 of the upper arm 26 includes or defines a fifth limiter, shown as first internal stop 202, positioned within the upper hinge interface 210. As shown in FIG. 11, the first internal stop 202 is positioned to engage with the first link 236 of the first connector 230 to limit an amount of extension of the knee brace 10. In some embodiments, the lower hinge interface 220 of the lower arm 36 additionally or alternatively includes or defines a sixth limiter positioned within the lower hinge interface 220. The sixth limiter may be positioned to engage with the second link 246 of the second connector 240 to limit an amount of extension of the knee brace 10. Such limiting of the extension of the knee brace 10 may assist in preventing hyperextension of the knee of the wearer of the knee brace 10.

As shown in FIGS. 11-12, the upper hinge interface 210 of the upper arm 26 includes or defines a seventh limiter, shown as second internal stop 204, positioned within the upper hinge interface 210. As shown in FIG. 12, the second internal stop 204 is positioned to engage with the second link 246 of the second connector 240 to limit an amount of flexion of the knee brace 10. In some embodiments, the upper hinge interface 210 of the upper arm 26 and/or the lower hinge interface 220 of the lower arm 36 additionally or alternatively include or define an eighth limiter. The eighth limiter may be positioned to engage with the first link 236 of the first connector 230 to limit an amount of flexion of the knee brace 10. Such limiting of the flexion of the knee brace 10 may assist in preventing hyper-flexion of the knee of the wearer of the knee brace 10.

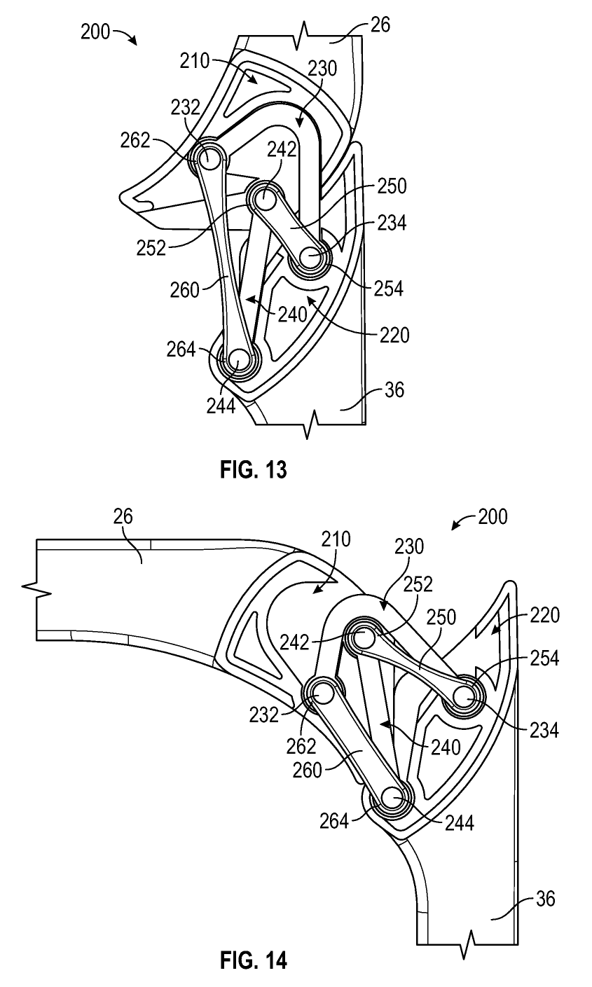

As shown in FIGS. 13-14, each of the four bar linkage hinges 200 includes a first resilient member, shown as flexion elastomer band 250. The flexion elastomer band 250 has a first end, shown as upper end 252, and an opposing second end, shown as lower end 254. The upper end 252 of the flexion elastomer band 250 is coupled to the upper end 242 of the second connector 240 and the lower end 254 of the flexion elastomer band 250 is coupled to the lower end 234 of the first connector 230 such that the flexion elastomer band 250 extends between the upper hinge interface 210 and the lower hinge interface 220.

As shown in FIGS. 13-14, each of the four bar linkage hinges 200 includes a second resilient member, shown as extension elastomer band 260. The extension elastomer band 260 has a first end, shown as upper end 262, and an opposing second end, shown as lower end 264. The upper end 262 of the extension elastomer band 260 is coupled to the upper end 232 of the first connector 230 and the lower end 264 of the extension elastomer band 260 is coupled to the lower end 244 of the second connector 240 such that the extension elastomer band 260 extends between the upper hinge interface 210 and the lower hinge interface 220.

As shown in FIG. 13, the four bar linkage hinge 200 is arranged in an extended orientation. In the extended orientation, the upper arm 26 of the upper frame member 20 and/or the lower arm 36 of the lower frame member 30 pivot about the four bar linkage hinge 200 such that front end of the upper hinge interface 210 (e.g., the first upper stop 216, etc.) and the front end the lower hinge interface 220 (e.g., the first lower stop 226, etc.) rotate towards each other, while the rear end of the upper hinge interface 210 (e.g., the second upper stop 218, etc.) and the rear end the lower hinge interface 220 (e.g., the second lower stop 228, etc.) rotate away from each other (e.g., in response to the wearer of the knee brace 10 extending/straightening his/her leg, etc.). Such rotation pulls on the extension elastomer band 260, while relaxing the flexion elastomer band 250. The pulling of the extension elastomer band 260 causes the extension elastomer band 260 to provide a biasing force against the rotation of the upper frame member 20 and/or the lower frame member 30 to rotationally bias the four bar linkage hinge 200 and the knee brace 10 back towards a nominal or neutral orientation (e.g., similar to the dual pivoting hinge 100 shown in FIG. 9, etc.). In the nominal or neutral orientation, the flexion elastomer band 250 and the extension elastomer band 260 may be in equilibrium (e.g., the flexion elastomer band 250 and the extension elastomer band 260 are not biasing the upper frame member 20 and/or the lower frame member 30 towards another orientation, etc.).

As shown in FIG. 14, the four bar linkage hinge 200 is arranged in a flexed orientation. In the flexed orientation, the upper arm 26 of the upper frame member 20 and/or the lower arm 36 of the lower frame member 30 pivot about the four bar linkage hinge 200 such that front end of the upper hinge interface 210 (e.g., the first upper stop 216, etc.) and the front end the lower hinge interface 220 (e.g., the first lower stop 226, etc.) rotate away from each other, while the rear end of the upper hinge interface 210 (e.g., the second upper stop 218, etc.) and the rear end the lower hinge interface 220 (e.g., the second lower stop 228, etc.) rotate towards each other (e.g., in response to the wearer of the knee brace 10 flexing/bending his/her leg, etc.). Such rotation pulls on the flexion elastomer band 250, while relaxing the extension elastomer band 260. The pulling of the flexion elastomer band 250 causes the flexion elastomer band 250 to provide a biasing force against the rotation of the upper frame member 20 and/or the lower frame member 30 to rotationally bias the four bar linkage hinge 200 and the knee brace 10 back towards the nominal or neutral orientation (e.g., similar to the dual pivoting hinge 100 shown in FIG. 9, etc.).

In one embodiment, the flexion elastomer band 250 and the extension elastomer band 260 have similar characteristics (e.g., spring constant, biasing force, etc.) such that the four bar linkage hinges 200 may bias the knee brace 10 similarly during extension and flexion. In other embodiments, the flexion elastomer band 250 and the extension elastomer band 260 have different characteristics (e.g., spring constant, biasing force, length, etc.) such that the four bar linkage hinges 200 may bias the knee brace 10 differently during extension and flexion. In some embodiments, the flexion elastomer band 250 and the extension elastomer band 260 are asymmetrically loaded to adjust the neutral position (e.g., the neutral angle, etc.) of the knee brace 10 in the neutral orientation. By way of example, the flexion elastomer band 250 and/or the extension elastomer band 260 may be configured such that the knee brace 10 is naturally biased toward a neutral orientation that is slightly flexed.

In some embodiments, the four bar linkage hinges 200 include a plurality of flexion elastomer bands 250 and/or a plurality of extension elastomer bands 260 to selectively adjust (e.g., increase, decrease, etc.) the biasing force during flexion and/or extension of the knee brace 10. In some embodiments, the four bar linkage hinges 200 do not include the flexion elastomer bands 250 such that the four bar linkage hinges 200 encourage flexion (i.e., the four bar linkage hinges 200 bias against extension, not flexion). In other embodiments, the four bar linkage hinges 200 do not include the extension elastomer bands 260 such that the four bar linkage hinges 200 encourage extension (i.e., the four bar linkage hinges 200 bias against flexion, not extension).

In some embodiments, one of the two four bar linkage hinges 200 (e.g., in a two hinge embodiment, etc.) of the knee brace 10 does not include the flexion elastomer band 250 and the extension elastomer band 260. For example, a four bar linkage hinge 200 positioned on the medial side of the knee brace 10 may not include the flexion elastomer band 250 and the extension elastomer band 260 such that the four bar linkage hinge 200 may have a lower profile (e.g., relative to the four bar linkage hinge 200 having the flexion elastomer band 250 and the extension elastomer band 260, etc.). The lower profile of the four bar linkage hinge 200 may provide the wearer of the knee brace 10 with closer contact with a vehicle (e.g., motorcycle, dirt bike, ATV, etc.) he/she is driving.

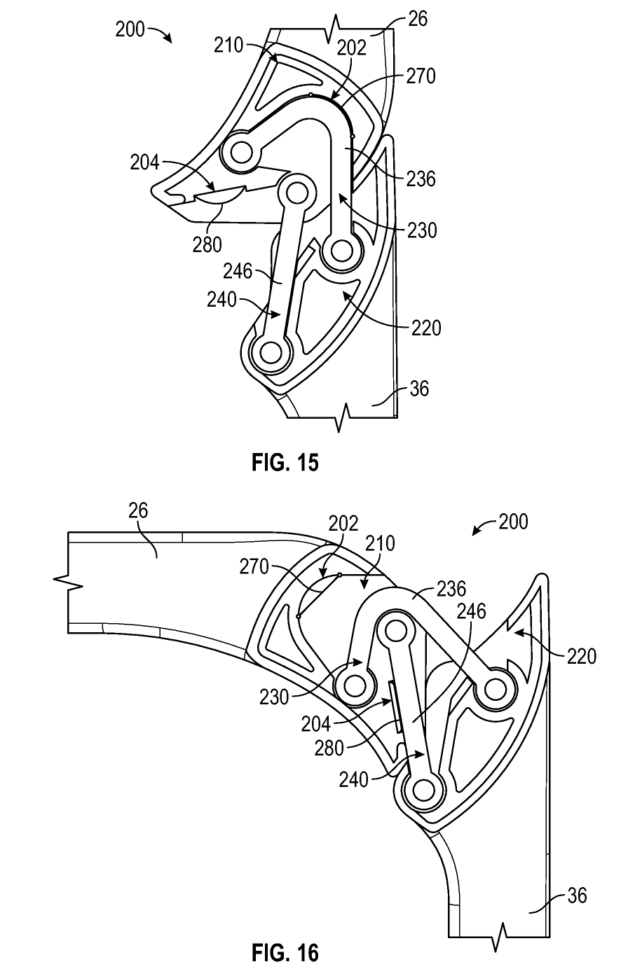

As shown in FIGS. 15-16, each of the four bar linkage hinges 200 includes a third resilient member, shown as first leaf spring 270. The first leaf spring 270 is positioned along the first internal stop 202 of the upper hinge interface 210. As shown in FIG. 15, the first leaf spring 270 is positioned along the first internal stop 202 to engage with the first link 236 of the first connector 230 when the knee brace 10 is arranged in the extended orientation. Such engagement between the first leaf spring 270 and the first link 236 may provide a biasing force against the rotation of the upper frame member 20 and/or the lower frame member 30 to rotationally bias the four bar linkage hinge 200 and the knee brace 10 back towards a nominal or neutral orientation.

As shown in FIGS. 15-16, each of the four bar linkage hinges 200 includes a fourth resilient member, shown as second leaf spring 280. The second leaf spring 280 is positioned along the second internal stop 204 of the upper hinge interface 210. As shown in FIG. 16, the second leaf spring 280 is positioned along the second internal stop 204 to engage with the second link 246 of the second connector 240 when the knee brace 10 is arranged in the flexed orientation. Such engagement between the second leaf spring 280 and the second link 246 may provide a biasing force against the rotation of the upper frame member 20 and/or the lower frame member 30 to rotationally bias the four bar linkage hinge 200 and the knee brace 10 back towards a nominal or neutral orientation.

In one embodiment, the first leaf spring 270 and the second leaf spring 280 have similar characteristics (e.g., spring constant, biasing force, size, etc.) such that the four bar linkage hinges 200 may bias the knee brace 10 similarly during extension and flexion. In other embodiments, the first leaf spring 270 and the second leaf spring 280 have different characteristics (e.g., spring constant, biasing force, size, etc.) such that the four bar linkage hinges 200 may bias the knee brace 10 differently during extension and flexion. In some embodiments, the four bar linkage hinges 200 do not include the second leaf spring 280 such that the four bar linkage hinges 200 encourage flexion (i.e., the four bar linkage hinges 200 bias against extension, not flexion). In other embodiments, the four bar linkage hinges 200 do not include the first leaf spring 270 such that the four bar linkage hinges 200 encourage extension (i.e., the four bar linkage hinges 200 bias against flexion, not extension). In some embodiments, one of the two four bar linkage hinges 200 (e.g., in a two hinge embodiment, etc.) of the knee brace 10 does not include the first leaf spring 270 and the second leaf spring 280.

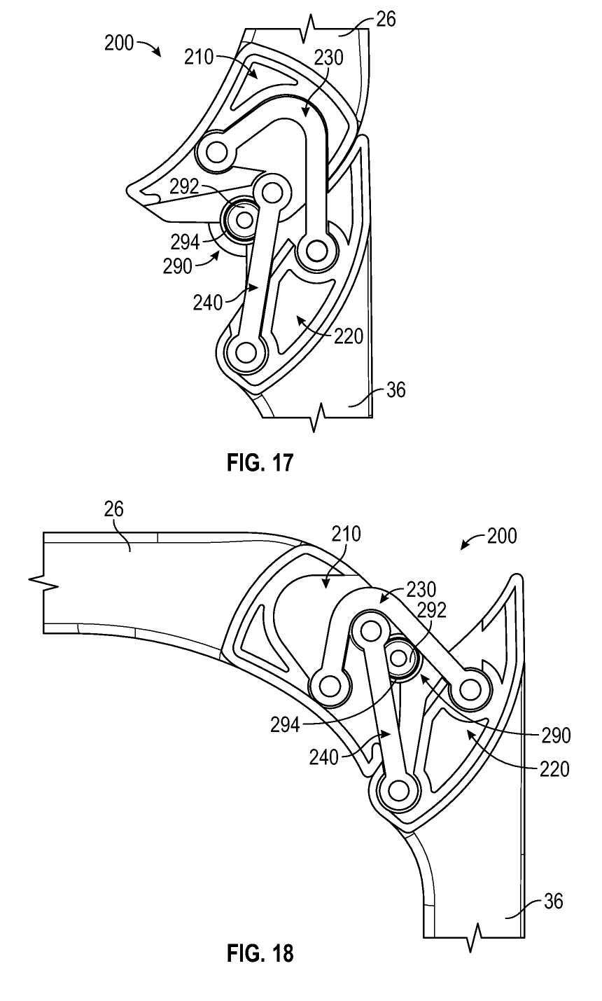

As shown in FIGS. 17-18, each of the four bar linkage hinges 200 includes an interface, shown as coupling interface 290. According to an exemplary embodiment, the coupling interface 290 rotationally couples the upper hinge interface 210 and the lower hinge interface 220. The coupling interface 290 includes a recess, shown as spring cavity 292, configured to receive a fifth resilient member, shown as torsion spring 294. According to an exemplary embodiment, the torsion spring 294 is configured to provide a biasing force against the rotation of the upper frame member 20 and/or the lower frame member 30 to rotationally bias the four bar linkage hinge 200 and the knee brace 10 from an extended orientation (e.g., see FIG. 17, etc.) and/or a flexed orientation (e.g., see FIG. 18, etc.) back towards a nominal or neutral orientation.

In one embodiment, the torsion spring 294 is configured to rotationally bias the four bar linkage hinges 200 in both extension and flexion. In other embodiments, the torsion spring 294 is configured to rotationally bias the four bar linkage hinges 200 only during extension (i.e., encourages flexion, does not bias against flexion). In still other embodiments, the torsion spring 294 is configured to rotationally bias the four bar linkage hinges 200 only during flexion (i.e., encourages extension, does not bias against extension). In some embodiments, the torsion spring 294 includes a first torsion spring configured to bias against flexion and a second torsion spring configured to bias against extension. In one embodiment, the first torsion spring and the second torsion spring have similar characteristics (e.g., spring constant, biasing force, size, etc.) such that the four bar linkage hinges 200 may bias the knee brace 10 similarly during extension and flexion. In other embodiments, the first torsion spring and the second torsion spring have different characteristics (e.g., spring constant, biasing force, size, etc.) such that the four bar linkage hinges 200 may bias the knee brace 10 differently during extension and flexion. In some embodiments, the first torsion spring and the second torsion spring are asymmetrically loaded to adjust the neutral position (e.g., the neutral angle, etc.) of the knee brace 10 in the neutral orientation. By way of example, the first torsion spring and/or the second torsion spring may be configured such that the knee brace 10 is naturally biased toward a neutral orientation that is slightly flexed. In some embodiments, one of the two four bar linkage hinges 200 (e.g., in a two hinge embodiment, etc.) of the knee brace 10 does not include the first torsion spring and the second torsion spring (e.g., a low profile hinge, etc.).

According to an exemplary embodiment, the four bar linkage hinges 200 include at least one of (i) the flexion elastomer band 250, (ii) the extension elastomer band 260, (iii) the first leaf spring 270, (iv) the second leaf spring 280, and (v) the torsion spring 294 (e.g., the first torsion spring and/or the second torsion spring, etc.). Any combination of the flexion elastomer band 250, the extension elastomer band 260, the first leaf spring 270, the second leaf spring 280, and the torsion spring 294 may be used to rotationally bias the four bar linkage hinges 200 and the knee brace 10 from an extended orientation and/or a flexed orientation to a nominal position or nominal orientation.

It is important to note that the construction and arrangement of the elements of the systems, methods, and/or apparatuses as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the enclosure may be constructed from any of a wide variety of materials that provide sufficient strength or durability, in any of a wide variety of colors, textures, and combinations.

Embodiments have been described in connection with the accompanying drawings. However, it should be understood that the figures are not drawn to scale. Distances, angles, shapes, etc. are merely illustrative and do not necessarily bear an exact relationship to actual dimensions and layout of the articles that are illustrated. In addition, the foregoing embodiments have been described at a level of detail to allow one of ordinary skill in the art to make and use the articles, parts, different materials, etc. described herein. A wide variety of variation is possible. Articles, materials, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or configurations are in any way required for one or more embodiments. The terms “comprising,” “including,” “having,” and the like are synonymous and are used inclusively, in an open-ended fashion, and do not exclude additional elements, features, acts, operations, and so forth. The term “consisting essentially of” can be used anywhere where the terms comprising, including, containing or having are used herein, but consistent essentially of is intended to mean that the claim scope covers or is limited to the specified materials or steps recited and those that do not materially affect the basic and novel characteristic(s) of the claimed invention. Also, the term “consisting of” can be used anywhere where the terms comprising, including, containing or having are used herein, but consistent of excludes any element, step, or ingredient not specified in a given claim where it is used.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, in the subject description, the word “exemplary” is used to mean serving as an example, instance, or illustration. Any embodiment or design described herein as “exemplary” is not necessarily to be construed as preferred or advantageous over other embodiments or designs. Rather, use of the word exemplary is intended to present concepts in a concrete manner. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. A knee brace, comprising:
an upper frame portion;
a lower frame portion;
a hinge pivotally coupling the upper frame portion and the lower frame portion; and
a knee cup including:
an outer cup having a first portion coupled to the upper frame portion and a second portion coupled to the lower frame portion; and
an inner cup having an outer surface and an inner surface, the inner surface positioned to engage a patella of a wearer of the knee brace.

2. The knee brace of claim 1, wherein the inner cup remains stationary relative to the outer cup as the upper frame portion and the lower frame portion pivot about the hinge.

3. The knee brace of claim 2, wherein the first portion of the outer cup rotates along the outer surface of the inner cup as the upper frame portion pivots about the hinge, and wherein the second portion of the outer cup rotates along the outer surface of the inner cup as the lower frame portion pivots about the hinge.

4. The knee brace of claim 1, wherein the first portion and the second portion of the outer cup enclose around the inner cup when the knee brace is arranged in an extended orientation, and wherein the first portion and the second portion of the outer cup separate to expose the inner cup when the knee brace is arranged in a flexed orientation.

5. The knee brace of claim 1, wherein the hinge includes a dual pivoting hinge.

6. The knee brace of claim 5, the upper frame portion is pivotally coupled to a first pivot of the dual pivoting hinge and the lower frame portion is pivotally coupled to a second pivot of the dual pivoting hinge.

7. The knee brace of claim 6, wherein the dual pivoting hinge includes at least one resilient remember extending laterally between the first pivot and the second pivot, the at least one resilient member positioned to bias the dual pivoting hinge towards a nominal position when the knee brace is arranged in at least one of a flexed orientation and an extended orientation.

8. The knee brace of claim 1, wherein the hinge includes a four bar linkage.

9. The knee brace of claim 1, wherein the hinge includes at least one of a first hinge positioned on a lateral side of the knee brace and a second hinge positioned on a medial side of the knee brace.

10. The knee brace of claim 1, wherein the knee cup further includes a patella immobilization elastomer positioned along the inner surface of the inner cup, the patella immobilization elastomer configured to assist in securing the patella of the wearer.

11. A knee brace, comprising:
a first frame portion;
a second frame portion; and
a dual pivoting hinge pivotally coupling the first frame portion and the second frame portion, the dual pivoting hinge including:
a first pivot coupling the first frame portion to the dual pivoting hinge;
a second pivot coupling the second frame portion to the dual pivoting hinge; and
a resilient member extending laterally between the first pivot and the second pivot.

12. The knee brace of claim 11, wherein the dual pivoting hinge is positioned on a lateral side of the knee brace.

13. The knee brace of claim 12, further comprising a second dual pivoting hinge positioned on a medial side of the knee brace.

14. The knee brace of claim 11, wherein the resilient member is positioned to bias the dual pivoting hinge towards a nominal position when the knee brace is arranged in at least one of a flexed orientation and an extended orientation.

15. The knee brace of claim 14, wherein the resilient member includes a first resilient member and a second resilient member, the first resilient member positioned to bias the dual pivoting hinge towards the nominal position when the knee brace is arranged in the flexed orientation, and the second resilient member positioned to bias the dual pivoting hinge towards the nominal position when the knee brace is arranged in the extended orientation.

16. The knee brace of claim 11, further comprising a nested knee cup including:
a first cup having a first portion coupled to the first frame portion and a second portion coupled to the second frame portion; and
a second cup having an outer surface and an inner surface, the inner surface positioned to engage a patella of a wearer of the knee brace and the outer surface positioned to engage the first cup;
wherein the second cup remains stationary relative to the first cup as the first frame portion and the second frame portion pivot about the dual pivoting hinge such that the first portion of the first cup rotates along the outer surface of the second cup and the second portion of the first cup rotates along the outer surface of the second cup, exposing the second cup.

17. A knee brace, comprising:
a first frame portion including a first extension, the first extension defining a first hinge interface;
a second frame portion including a second extension, the second extension defining a second hinge interface;
a first coupler extending between the first hinge interface and the second hinge interface; and
a second coupler extending between the first hinge interface and the second hinge interface;
wherein the first hinge interface, the second hinge interface, the first coupler, and the second coupler cooperatively form a four bar linkage that pivotally couples the first frame portion and the second frame portion.

18. The knee brace of claim 17, wherein the first extension includes a first stop and the second extension includes a second stop, the first stop and the second stop positioned to engage to limit extension of the knee brace.

19. The knee brace of claim 18, wherein the first extension includes a third stop and the second extension includes a fourth stop, the third stop and the fourth stop positioned to engage to limit flexion of the knee brace.

20. The knee brace of claim 17, further comprising a resilient member positioned to bias at least one of the first frame portion and the second frame portion towards a nominal position, the resilient member including at least one of (i) an elastomer band, (ii) a leaf spring, (iii) and a torsion spring.
